(19) United States
(12) Patent Application Publication
(10) Pub. No.: US 2008/0092902 A1
(43) Pub. Date: Apr. 24, 2008

(54) Device For Supplying Inhaled Air

(76) Inventor: Ralf Schnell, Seligenstadt (DE)

Correspondence Address: PAUL AND PAUL, 2000 MARKET STREET, SUITE 2900, PHILADELPHIA, PA, 19103, US

(21) Appl. No.: 11/816,786

(22) PCT Filed: Feb. 24, 2006
(86) PCT No.: PCT/EP06/60282

§ 371 Date: Oct. 24, 2007

(30) Foreign Application Priority Data
Feb. 28, 2005 (DE) 10 2005 009 577.1
Mar. 31, 2005 (DE) 10 2005 015 015.4

(51) Int. Cl. A61M 16/04

(52) U.S. Cl. 128/207.15

(57) ABSTRACT

The invention relates to an inhaled air supplying device, in particular, an endotracheal tube or a tracheostoma cannula comprising of at least one tube (1) and at least one cuff (2) which is fillable with a fluid and arranged outside of the tube for sealing said tube against an air conduit wall (10). In order to provide inhaled air supplying devices devoid of known disadvantages, the invention is characterised in that the cuff comprises, at least sectionally, a wear-resistant surface layer (6, 7) whose thickness is less than 10 μm and which modifies the surface of the device material.

The present invention relates to a device for supplying inhaled air with at least one tube.

Various types of devices for supplying inhaled air are known from the state of the art. Such devices are used primarily as so-called endotracheal tubes for the artificial respiration of patients in intensive care and surgery. The endotracheal tube is passed through the nose or mouth of the patient into the trachea or upper bronchial area.

In addition, such devices are used as so-called tracheostomy cannulae for the respiration of patients with a tracheotomy. These tracheostomy cannulae can also be used for the permanent autonomous respiration of the patient, e.g. after laryngotomies. The tracheostomy cannulae are passed through the tracheotomy into the trachea where they likewise extend into the upper bronchial area.

Each of the tubes forms a continuous lumen which connects the bronchial area of the patient with the ambient air or else with a respirator.

In most cases the devices known from the state of the art are composed of various plastics which are flexible and can be produced cheaply under sterile conditions. However, the disadvantage of the tubes known from the state of the art is that they have a high coefficient of friction due to the nature of the plastic materials. This is disadvantageous in particular during intubation, i.e. the passing of the tube through the mouth, nose or through the tracheotomy. Doctors and orderlies must exert considerable force during intubation as a result of the increased friction between the devices and the organs concerned of the patient. In addition, the organs concerned can be damaged as a result of the friction.

The plastic materials used to produce the tubes rapidly accumulate contaminants, in particular secretions and food particles mixed with saliva, and their surface becomes soiled. The tubes stick together or become encrusted, in particular in the case of long-term respiration via the tubes. The contaminants that accumulate on the surface of the tubes form a breeding-ground for bacterial infections of the organs of the respiratory tract.

A common complication in the respiration of patients via tubes of the type described above is so-called aspiration. Because the protective reflexes are switched off by the tubes that are passed into the respiratory tract, liquid or solid substances penetrate into the respiratory tract, resulting in shifts in the respiratory tract and consequently in hypoxias. In addition, the impurities in the area of the bronchial tubes form breeding-grounds for bacteria and germs, with the result that when tubes are used for respiration pneumonia is very often one of the complications following respiration.

In order to prevent aspiration, i.e. the penetration of liquid and solid substances into the bronchial area, when supplying inhaled air, so-called “cuffs” are known from the state of the art, for example from EP 0 930 909 B1. These are flexible cuffs that can be inflated or filled with another fluid which are provided on the outside of the tube and are in general securely connected to same. The cuffs are attached to the tube such that they come to rest in the upper area of the trachea. In the inflated or filled state, they fill the trachea outside the tube and seal the outside of the tube off from the wall of the trachea and thus prevent liquids or solid substances, e.g. food particles, from reaching the bronchial area through the lumen between the outer wall of the tube and the wall of the gullet.

The low-pressure cuffs known from the state of the art have certain disadvantages because, in order to have a sealing effect, they must have a diameter which must be basically larger than the internal diameter of the trachea. When the cuff is filled, folds form in the cuff material which extend inwards from the outer periphery of the cuff. These folds form through-channels through which liquids can enter the bronchial area.

It has been shown that the folds extend mainly in longitudinal direction of the cuff, wherein folds can also cross over or stick to each other, which at any rate means that small gaps thereby form between these fold walls which do not completely close due to the limited ductility of the film material, even if it is highly flexible. This means that so-called “fold loops” form, in particular at the radial inner end of the folds, which have a certain permeability for liquids and thus also for the secretions accumulating in the trachea, which can thus enter the lung and in so doing very often cause pneumonia. Also forming at the transition from such folds to the wall of the trachea are approximately triangular gussets which define passage openings or channels between trachea and cuff and along the outside of the cuff.

For this reason, there is a trend towards cuffs with even greater flexibility in order to avoid such passage channels or to make them even narrower and thus more impermeable, which is achieved in general by reducing the wall thickness of the cuff, wherein films with wall thicknesses of only 5 μm are already being used.

However, it does happen that the cuffs fill with water/secretion. The thinner the cuffs are, the more rapidly the liquid can pass (diffuse/migrate) through the cuff wall. This is the case even when the cuffs possibly form very narrow folds through which hardly any secretion can still pass.

Added to this is the problem that the extremely thin-walled cuff material is difficult to handle and can also be easily damaged.

Compared with this state of the art, the object of the present invention is to provide devices for supplying inhaled air which avoid the above-named disadvantages.

This object is achieved according to the invention in that a device for supplying inhaled air with at least one tube and at least one cuff that can be filled with a fluid and provided on the outside of the tube to seal off the tube from the trachea wall is provided, wherein at least the cuff has at least in sections an abrasion-resistant surface layer (6, 7), which modifies the surface of the material of the device, with a layer thickness of less than 10 μm.

In this way, the advantages of the basic material used, such as e.g. flexibility and cheap production, are retained, while the surface properties of the plastic elements of the device are modified in a targeted manner and adjusted according to the application with the help of suitable formation of the surface layer, in particular by coating.

The cuff is expediently a so-called “high volume low pressure” cuff. This is characterized in that it has only a small wall thickness and the outer diameter of the cuff balloon is greater than the diameter of the trachea. When filling, the cuff therefore adapts to the contours of the trachea, accompanied by the formation of folds. A sealing by these cuffs is possible even at relatively low pressures (typically 20 millibars). Because of the low pressures, damage to the trachea is avoided.

It is advantageous in particular if the cuff is composed of a flexible, thin-walled plastic film. Embodiments in which the plastic film surrounds the tube in the shape of a widened section of hose are preferred. An embodiment of the invention in which the film has a water-impermeable layer on at least one of its sides is particularly preferred.

Coating with a water-impermeable material makes it possible to still use film material for the cuff which is thin-walled, highly flexible and forms extremely small fold gaps, and which yet for its part is not water- or moisture-permeable. The term “water-impermeable”, which is also to include “moisture-impermeable”, is naturally to be interpreted in the given technical context, i.e. a certain residual, minimum water permeability, which can be tolerated for practical use, can still be present. It is essential however that the water permeability e.g. of a soft polyurethane film of 5 μm wall thickness is reduced to a fraction of less than ⅓, preferably less than 1/10 or even less than 1/100. In the case of a water-impermeable layer, in contrast, the thickness of the layer does not matter, with the result that this can also be thicker than 10 μm, wherein the abrasion resistance of the layer merely represents a preferred embodiment.

The film material can be selected from a group comprising polyurethane, polyester, PET and PVC, wherein polyurethane is most preferred because firstly it is a very tissue-compatible material and secondly can be produced in a very flexible and ductile form. The more flexible and ductile the film material is, the smaller are the resulting fold loops or gussets at the transition from the fold of a cuff to the trachea for a given wall thickness. Polyurethane can be produced for example in various hardnesses. However, particularly soft materials have the disadvantage that they also lose tensile strength, with the result that in this respect there must be a compromise between tensile strength and ductility. The disadvantage of the relatively high water- or moisture-permeability of thin-walled (and where possible also soft) polyurethane is offset without difficulty by the measures according to the invention.

It is expedient if the cuff is coated with the water-impermeable material on its outside facing away from the tube.

The film should expediently have a wall thickness of less than 100 μm, wherein on the other hand wall thicknesses of more than 5 μm are also preferred for practical reasons. Wall thicknesses of 10 μm to 50 μm have proved suitable in practice, wherein a wall thickness of the film in the range from 15 to 30 μm is most preferred for the present invention.

In addition, it is provided in a particularly preferred embodiment of the present invention that an additional, hydrophobing layer is applied to the outside of the cuff or film material of which the cuff is composed. Such an additional, hydrophobing layer is applied e.g. to a previously applied water-impermeable coating or else the water-impermeable coating is located on the other side of the film which serves as the inside of the cuff.

Ideally, coating materials can also be found which have a good tissue compatibility, adhere well to the film material, in particular polyurethane, and finally are also both water-impermeable and hydrophobic.

A wide variety of forms of materials is available which can be considered as water-impermeable coatings. In particular, numerous inorganic coatings are suitable for this, such as e.g. metals or silicon dioxide, and on the other hand also organic coatings, such as e.g. fluorinated hydrocarbons such as Teflon or silane. If the coating takes place on the inside of the cuff or else is covered by an additionally applied hydrophobic layer, tissue compatibility or body compatibility also plays only a small part, although in principle the use of body-compatible and non-toxic coating materials is still preferred even if they do not as a rule come into contact with body parts.

In particular the combination of a water-impermeable with a hydrophobic coating has the advantage that reliance on extremely thin-walled films in the region of 5 μm or even less is not necessary since, because of the hydrophobic coating, liquid secretion is prevented from passing through the gaps formed by the folds even if these are somewhat larger, as seems unavoidable when using correspondingly thicker films.

Film-wall thicknesses of the order of approx. 25 μm (more precisely in the range from 15 to 30 μm) have proved very easy to handle and, with a corresponding, combined coating with a water-impermeable and a hydrophobic material, lead to cuffs which have the most favourable properties compared with all known cuffs, which affects their manageability and safety on the one hand (for example against unintended damage), but also their function on the other. These cuffs are thin-walled and flexible enough to lie tight against the wall of the trachea when there is a slight excess pressure, wherein the resulting folds are sufficiently small to prevent the passage of secretion through the fold gaps, at least on account of the additional hydrophobic coating.

Both the water-impermeable coatings and the hydrophobic coatings can be applied in very small layer thicknesses which do not adversely affect the mechanical properties of the films and in particular their flexibility and ductility. The thickness of the water-impermeable layer is preferably less than 5 μm, in particular less than 1 μm and particularly preferably less than 200 nm. The same also applies to the hydrophobic coating, with the result that if each of the two layers is for example only approximately 100 nm thick, the overall thickness of the coatings accounts for only 1% in a film with a wall 20 μm thick and can thus be disregarded for the mechanical properties of the film.

Above all for example a hydrophobic surface layer of the previously described cuffs prevents, despite the formation of some folds, the secretion which accumulates above the cuff from penetrating into or through the unavoidable folds of the cuffs even if, due to the maximum clear width of these folds, the secretion would flow easily along these folds past the cuff if its surface were not composed of a particular material, in particular a hydrophobic material.

A special surface layer is also expedient in the case of a cuff composed of an elastic material which expands upon filling and fits the contour of the trachea in optimum manner without forming folds. As such a cuff also may not exert a strong pressure on the wall of the trachea and there is thus also the latent risk of a leak, the tightness of such a cuff in the trachea can be further improved by the adapted surface layer.

An embodiment of the invention in which the surface layers are thin, in particular have a thickness of less than 5 μm, preferably less than 3 μm and particularly preferably less than 1 μm, is preferred. Very thin layer thicknesses of even less than 500 or 200 nm are easily sufficient for decisive modifications of the surfaces. Layer thicknesses of less than 100 nm or even monomolecular or monatomic layers only a few nm thick which define the lower limit of layer thickness at approximately 2 to 5 nm are often sufficient for this purpose. Thin surface layers have the advantage that they leave the flexibility and/or elasticity of the basic material substantially unchanged. This is advantageous in particular with the above-named “high volume low pressure” cuffs in which the material of the cuff is already in most cases less than 50 μm thick.

The claimed abrasion resistance is to obtain primarily during a normal use when touching, handling, sterilizing and inserting. It obtains in particular in the case of a chemical or physical bonding of the surface layer to the plastic material of the device. This property is intended to distinguish the surface layers according to the invention primarily from creams or gels and also swellable coatings which are applied to the surface of the tubes with a relatively low adhesion so that when handling them measures must be taken to ensure that the integrity of the coating is not impaired. Creams or gels, for example, can be wiped or rubbed off again after application and, at least largely locally, removed. These disadvantages are to be avoided by the abrasion resistance.

The above-named abrasion-resistant surface layers can currently be cheaply and economically produced in various ways using thin-layer technology.

An embodiment of the invention in which the surface layer is a plasma coating is particularly preferred.

If the surface layer or coating is a polymer, the polymer can already be added to the basic material during the production process. If polymers or copolymers of the plastic material of the device are used for this which have a lower surface tension than the plastic material, an accumulation of the added polymer or copolymer on the surface already occurs in the melt. In this way, thin surface layers with the surface properties of the polymer or copolymer are formed.

In addition it is advantageous if the surface layer has a reduced coefficient of friction compared with the material of the device. This makes it possible to introduce the device into the organs of the respiratory tract of the patient with reduced force, thereby also minimizing in particular the risk of injury to the patient.

In a further advantageous version, the device according to the invention has a silver-containing coating which in the simplest case is produced for example by vapour-plating with elemental silver. Alternatively, silver or silver ions could be incorporated into a coating material, in particular into hydrophobic coating material. Silver is known to have an antibacterial action which can be exploited in this manner. A vapour-plated silver layer can be so thin that it does not adversely affect the hydrophobic properties of a hydrophobic layer lying underneath it. The hydrophobicity of the surface layer is thus also retained with a silver vapour-plating.

In a preferred embodiment of the invention, the surface layer is dirt-repellent, with the result that no dirt deposits which could serve as a breeding-ground for bacteria can accumulate on the surface of the device, i.e. in particular of the tube and/or cuff.

An embodiment of the invention in which the surface layer is self-cleaning is particularly preferred. Such self-cleaning surface layers are known for example from structural engineering where they make it possible to significantly reduce the need to clean glass surfaces. In addition to the selection of the suitable surface-layer material, it can be necessary with such a self-cleaning surface layer to provide the surface with a microstructuring. This can be carried out for example through a process in which the molecules provided as a coating or surface layer order themselves on the surface of the tube or cuff.

An embodiment of the invention in which the surface-layer material is a fluorine-containing, e.g. CF₄, C₂F₆ among others, or a silicone-containing material is preferred. There may be named here by way of example coatings or surface layers which are produced using silanes, e.g. tetramethylsilane, siloxanes or their polymers.

The coatings can also be anchored to the surface of the plastic material of the device by chemical bonds (grafting).

The surface layers according to the invention modify not only the surface properties of the device in the desired manner, but also form e.g. a diffusion barrier for any additives such as plasticizers and similar contained in the basic material of the tube or cuff. In this way, smaller quantities of these substances enter the organism of the patient.

The previously named surface properties of the surface-layer materials can be expedient either individually or in combination.

An embodiment of the invention in which the cuff and the tube are coated, wherein these coatings can differ from one another, is preferred. However, it can alternatively be expedient if only one of the elements or selected sections thereof are coated. A coating of the inner walls of the tube or cuff is also possible in order for example to prevent encrustation of the tube and/or sticking of the walls of the cuff.

An embodiment of the invention in which the device is either a tracheostomy cannula, an endotracheal tube, a larynx mask or a pharyngeal tube is particularly preferred.

Further features, advantages and applications of the present invention become clear with reference to the following description of a preferred embodiment and the associated figures.

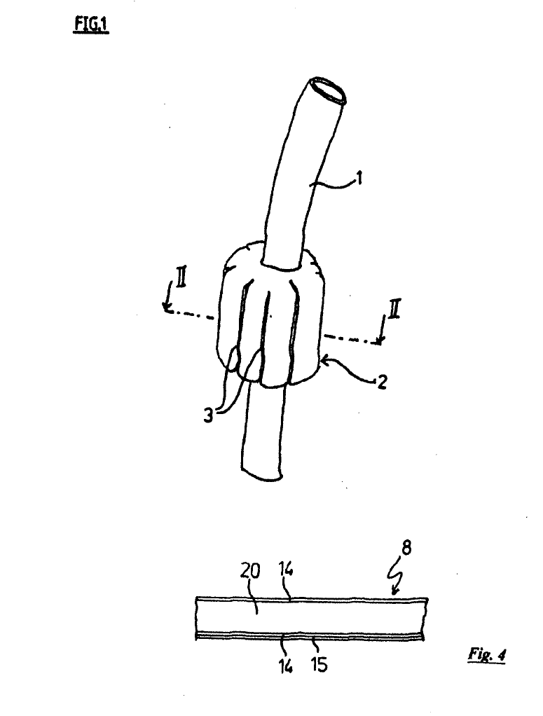

FIG. 1 shows schematically a device according to the invention with an inflated cuff.

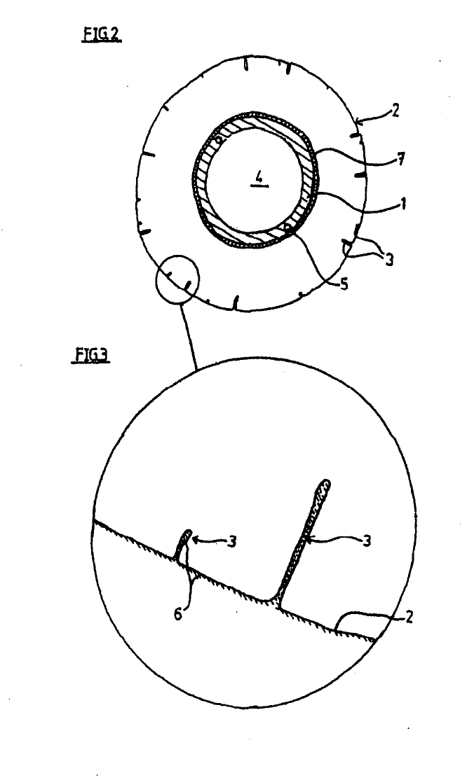

FIG. 2 shows a section view looking along the axis of the tube and with a section plane which runs approximately along the dotted line denoted II-II in FIG. 1.

FIG. 3 shows an enlarged cut-out section corresponding to the circle denoted III in FIG. 2.

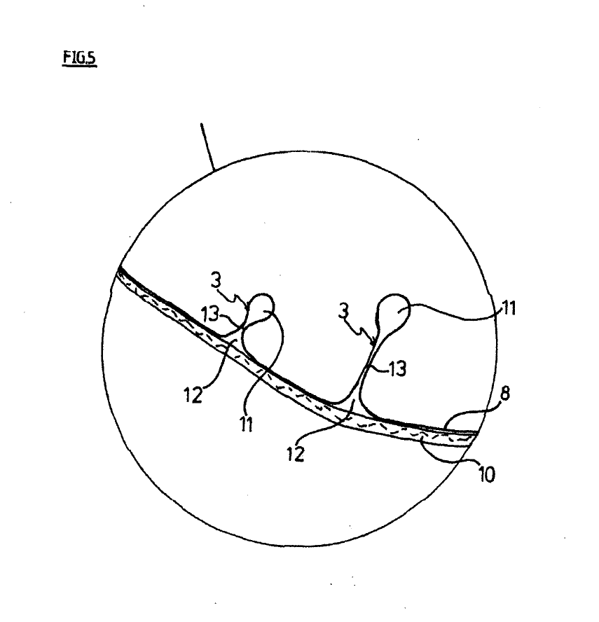

FIG. 4 shows a further enlarged section through the wall of the film material of the cuff.

FIG. 5 shows an enlarged cut-out section corresponding to the circle denoted III in FIG. 2, wherein contact with the trachea is shown.

FIG. 1 shows a tracheostomy cannula which consists essentially of a hose or a tube 1 on the outer periphery of which an inflatable cuff 2 is provided in a central section. The cuff 2 is welded securely to the tube. Alternatively, however, it can also be glued to or produced in one piece with the tube 1. A channel not visible here provided in the wall of the tube 1 has an opening in the outer wall of the tube 1 in the area of the cuff 2, with the result that the cuff 2 can be inflated, but optionally also deflated, via this outlet of the channel provided in the wall of the tube 1. Respiration takes place via the central lumen 4 of the tube 1.

FIG. 2 shows in section the tube in the area of the cuff 2. The hatched wall of the tube 1, a central lumen 4 and two wall channels 5 provided in the wall of the tube 1 which serve to inflate or deflate the cuff can be seen. Alternatively, one of the wall channels 5 can also serve to supply or evacuate liquid or secretion if it opens from the tube 1 into the trachea.

The cuff is shown in the filled state in the figures. In this state, the thin wall of the cuff rests against the trachea of the patient. Several longitudinal folds 3 are formed. However, FIGS. 1 to 3 show the subject of the invention only very schematically and also the formation of the folds 3, which can be seen in all three figures, is here reproduced only schematically. The folds can have any shape, wherein they do not necessarily extend over the whole length of the cuff.

In FIG. 5, the film of which the cuff 2 is composed is numbered 8 to distinguish it better. FIG. 5 shows, greatly enlarged and again only schematically, how the film 8 of the cuff 2 rests against the wall 10 of a trachea.

While the film 2 rests smooth and tight against the wall 10 of the trachea in the fold-free area, in the area of an unavoidable fold 3 there are primarily two critical passages which are numbered 11 and 12. 11 denotes a fold loop which forms at the inner end or base of a fold 3 when the film material is folded back in a loop, with the result that the outer walls of the film rest against each other at a distance from the base of the fold, as shown in area 13, but where the film 2 forms an 180° loop at the base of the fold the elastic restoring forces acting in the film prevent a pronounced kink from forming, with the result that the fold loop 11, shown only schematically here, is formed. Here, due to the elastic restoring forces acting in it, the film 8 assumes only a limited bending radius which, in the case of most preferred film materials, such as e.g. polyurethane, is six to fifteen times the wall thickness of the film material, at any rate in the case of the forces acting specifically here. A similarly problematic zone is however also the gusset area 12 which forms where the film 8 leaves the wall of the trachea to form the fold and nestles against the trachea again on the other side of the fold. It is to be taken into account that only a slight excess pressure of approximately 20-30 millibars is applied to the inside of the cuff, which is selected such that although the film 8 rests sealed-off against the wall 10 of the trachea, the blood circulation in the tracheal wall 10 is not to be impaired. Consequently, correspondingly small forces which are not sufficient to overcome the elastic restoring forces also act on the wall of the cuff in the area of the folds, with the result that the shown geometric relationships thereby approximately result even if the representation chosen here is not necessarily true to scale.

In the shown embodiment, the surface of the cuff is coated with a fluorine-containing compound, C₂F₆. A thin, polymeric hydrocarbon layer (CₓF_{y}H_{z})ₙ forms which is covalently bonded to the cuff surface. This coating is hydrophobic, with the result that no liquids or solids wetted with liquids can penetrate into and through the formed folds. Due to the high surface tension of many liquids, it is made much more difficult for these liquids to penetrate into narrow folds. In this way, the sealing action of the cuff is significantly improved and an aspiration can be effectively prevented, as neither liquids nor solids can enter the lower respiratory tract. Coating with C₂F₆ is carried out with the help of a plasma coating process.

As shown in the section view from FIG. 2, the tube 1 is likewise provided with a surface layer 7 which modifies the surface properties. This coating 7 is composed of a hydrophobic and dirt-repellent polymer.

The coating 7 in the shown embodiment of the tube 1 is a polysilane. It was added to the plastic material during the extrusion of the tube body and accumulated on the outer surface of the tube 1 due to its lower surface tension compared with the tube material. A thin surface layer forms there on the inner and outer lumen.

To make representation actually possible, the thickness of the surface layers 6, 7 shown in FIGS. 2 and 3 is not true to scale. While the hydrophobic, dirt-repellent coating 7 of the tube 1 has a thickness of approximately 3 μm, the coating 6 of the cuff 2 is preferably an atomic monolayer and if possible only a few nm, and in particular less than 100 nm, thick.

FIG. 4 shows schematically a section through the wall of a film 8, wherein the actual film material is shown here as a layer 20 which can have e.g. a thickness between 15 and 30 μm, wherein firstly a water-impermeable layer 14 and in addition a hydrophobic layer 15 are applied to the outside of the film which is shown at the bottom of FIG. 4.

The thickness of the layers 14 and 15 is in fact much smaller relative to the thickness of the actual film or support material 20 than is shown here.

This water-impermeable layer 14 can also be applied to the inside of the film material 20 optionally or alternatively to the water-impermeable layer 14 applied underneath the hydrophobic layer 15.

The present invention succeeds, despite a diameter of the fold loops 11 in the range from approximately 150-300 micrometres and despite corresponding dimensions in the gusset area 12, in preventing secretion from passing through, due to a hydrophobic coating 15 which is applied to the film 2 in addition to a water-impermeable coating 14.

However, in principle there is nothing to prevent film materials and wall thicknesses from being used which form even smaller fold loops 11 or gusset areas 12 in order to thereby more effectively prevent the passage of secretion.

The water-impermeable coating 14 simultaneously also sees to it that no water or secretion liquid whatever passes through the wall of the film 2.

For purposes of original disclosure, it is pointed out that all of the features revealed to a person skilled in the art by the present description, drawings and claims, even if they are described specifically only in connection with specific further features, can be combined both individually and in any combinations with other features or feature groups disclosed here, provided this has not been expressly excluded or technical circumstances make such combinations impossible or pointless. The comprehensive, explicit representation of all the conceivable feature combinations is dispensed with here only for the sake of brevity and readability of the description.

### LIST OF REFERENCE NUMBERS

1 endotracheal or tracheotomy tube
2 cuff
3 folds, longitudinal channels
4 central lumen
5 wall channels
6, 7 surface layers
8 film
10 wall of a trachea
11 fold loop
12 gusset area
13 are of adjacent outer walls

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. Device for supplying inhaled air, in particular endotracheal tube or tracheostomy cannula, comprising at least one tube and at least one cuff that can be filled with a fluid and that is provided on the outside of the tube to seal off the tube from the trachea wall, wherein at least the cuff has in sections an abrasion-resistant surface layer with a layer thickness of less than 10 μm which modifies the surface of the material of the device, and wherein the cuff comprises a flexible, thin-walled plastic film having a wall thickness of less than 100 μm (0.1 mm), wherein the thickness of the surface layer is less than 1 μm.

33. Device according to claim 32, wherein the thickness of the surface layer is between 0.05 μm and 0.5 μm.

34. Device according to claim 32, wherein the plastic film has a wall thickness of more than 5 μm.

35. Device according to claim 32, wherein the plastic film surrounds the tube in the shape of a widened section of hose and is secured to same.

36. Device according to claim 32, wherein the film has a water-impermeable layer on at least one of its sides.

37. Device according to claim 32, wherein the plastic film surrounds the tube in the shape of a widened section of hose and is secured to same.

38. Device according to claim 32, wherein the water-impermeable coating is composed of a metal which is selected from the group of the following substances or combinations thereof: silicon dioxide, metals, metal oxides or other inorganic coatings, silanes, siloxanes and fluorinated hydrocarbons, such as Teflon.

39. Device according to claim 32, wherein the cuff is coated with the water-impermeable material on its outside facing away from the tube.

40. Device according to claim 32, wherein the surface layer is both water-impermeable and hydrophobic.

41. Device according to claim 32, wherein an additional hydrophobing layer is applied to the outside of the cuff.

42. Device according to claim 32, wherein an additional hydrophobing layer is applied to the outside of the cuff and wherein the hydrophobing layer is composed of a material which is selected from the group comprising fluorinated hydrocarbons, silanes or siloxanes.

43. Device according to claim 32, wherein the material of the film of which the cuff is composed is selected from the group comprising polyurethane, PVC, SEBS and silicone.

44. Device according to claim 32, wherein the wall thickness of the film is between 10 and 50 μm.

45. Device according to claim 32, wherein the wall thickness of the film is between 15 and 30 μm.

46. Device according to claim 32, wherein the surface layer is both water-impermeable and hydrophobic.

47. Device according to claim 32, wherein the cuff coating is a plasma coating.

48. Device according claim 32, wherein the surface layer is produced by surface segregation of an additive contained in the cuff material, in particular polymer, preferably fluorine-containing polymers, silanes or siloxanes or their copolymers with the cuff material.

49. Cuff according to claim 32, wherein the cuff material contains inorganic particles such as preferably SiO₂, BaSO₄ or TiO₂ which act as a diffusion barrier or reduce the free volume in the polymer and thus reduce the diffusion/migration of molecules such as e.g. water.

50. Device according to claim 32, wherein the surface layer is hydrophobic.

51. Device according to claim 32, wherein the surface layer has a reduced coefficient of friction compared with the material of the device.

52. Device according to claim 32, wherein the surface layer is dirt-repellent and/or self-cleaning.

53. Device according to claim 32, wherein the surface layer is anchored to the device by chemical or physical bonding.

54. Device according to claim 32, wherein the surface layer is a plasma coating.

55. Device according to claim 32, wherein the surface layer contains fluorine.

56. Device according to claim 32, wherein the surface layer contains silicone, silane or siloxane.

57. Device according to claim 32, wherein the surface layer is CF₄, C₂F₆, a silane, preferably tetramethylsilane, or a siloxane, or has a corresponding polymer.

58. Device according to claim 32, wherein the surface layer is produced by segregation of an additive, in particular a copolymer, on the surface of the tube or cuff material.

59. Device according to claim 32, wherein the surface layer contains silver or silver ions.

60. Device according to claim 32, wherein the surface layer is vapour-plated with silver.

61. Device according to claim 32, wherein the cuff is composed of an intrinsically hydrophobic polymer material.

62. Device according to claim 32, wherein the tube is also coated.

63. Device according to claim 32, wherein it is a larynx mask.

64. Device for supplying inhaled air, in particular endotracheal tube or tracheostomy cannula, comprising at least one tube and at least one cuff that can be filled with a fluid and that is provided on the outside of the tube to seal off the tube from the trachea wall, wherein at least the cuff has in sections an abrasion-resistant surface layer with a layer thickness of less than 10 μm which modifies the surface of the material of the device, and wherein the cuff is composed of a flexible, thin-walled plastic film having a wall thickness of less than 100 μm (0.1 mm), wherein the thickness of the surface layer is less than 1 μm, and wherein the film has a water-impermeable layer on at least one of its sides.

65. Device according to claim 64, wherein the plastic film surrounds the tube in the shape of a widened section of hose and is secured to same.

66. Device according to claim 64, wherein the water-impermeable coating is comprised of a metal which is selected from the group of the following substances or combinations thereof: silicon dioxide, metals, metal oxides or other inorganic coatings, silanes, siloxanes and fluorinated hydrocarbons, such as Teflon.

67. Device according to claim 64, wherein the cuff is coated with the water-impermeable material on its outside facing away from the tube.

68. Device according to claim 64, wherein the surface layer is both water-impermeable and hydrophobic.

69. Device according to claim 64, wherein the cuff coating is a plasma coating.

70. Device for supplying inhaled air, in particular endotracheal tube or tracheostomy cannula, comprising at least one tube and at least one cuff that can be filled with a fluid and that is provided on the outside of the tube to seal off the tube from the trachea wall, wherein at least the cuff has in sections an abrasion-resistant surface layer with a layer thickness of less than 10 μm which modifies the surface of the material of the device, and wherein the cuff is comprised of a flexible, thin-walled plastic film having a wall thickness of less than 100 μm (0.1 mm) and more than 5 μm (0.005 mm), wherein the thickness of the surface layer is less than 1 μm.

71. Device according to claim 70, wherein the wall thickness of the film is between 10 and 50 μm.

72. Device according to claim 70, wherein the wall thickness of the film is between 15 and 30 μm.
